(19) United States
(12) Patent Application Publication
(10) Pub. No.: US 2005/0277913 A1
(43) Pub. Date: Dec. 15, 2005

(54) Heads-up display for displaying surgical parameters in a surgical microscope

(76) Inventor: Brian Douglas McCary, Clayton, MO (US)

Correspondence Address: Bausch & Lomb Incorporated, One Bausch & Lomb Place, Rochester, NY, 14604-2701, US

(21) Appl. No.: 10/864,052

(22) Filed: Jun. 9, 2004

(51) Int. Cl. A61B 17/00

(52) U.S. Cl. 606/1

(57) ABSTRACT

An ophthalmic surgical system 70 includes a surgery-viewing device 10 for observing a surgical site 72. A surgical console 74 controls at least one surgical instrument 64. The surgical console 74 detects certain surgical parameters during surgery. A heads-up display 12 is connected to each of the surgery-viewing device 10 and the surgical console 74 for displaying at least one of the surgical parameters to a user through the surgery-viewing device 10.

### BACKGROUND OF THE INVENTION

1. Field of the Invention

The present invention relates to heads-up displays. More specifically, the present invention relates to ophthalmic surgical systems wherein a heads-up display is connected to a surgical console and to a surgery-viewing device for displaying surgical parameters from the surgical console to the surgery-viewing device.

2. Description of Related Art

Presently, ophthalmic surgical systems typically have a display, such as an LCD screen or other general used display screen connected to a surgical console. These screens display many important surgical parameters to a user, such as phacoemulsification energy level, vacuum level, or other settings of the surgical equipment, which the surgeon must be aware of. In addition, a surgeon typically performs ophthalmic surgery through the use of a surgery-viewing device, such as a surgical microscope.

The microscope allows the surgeon to properly view the relatively small surgical site so that safe and efficient surgery on a patient's eyes, such as removing a cataract, may be achieved.

However, in order for the surgeon to view the various surgical parameters he must move his gaze from the surgical microscope to the surgical console display screen or listen for audible signals, which may be distracting to the surgeon. Therefore, it would be advantageous to supply the surgeon with needed surgical parameter information during surgery without requiring the surgeon to divert his attention and gaze from the surgical viewing device or microscope.

### BRIEF DESCRIPTION OF DRAWINGS

FIG. 1 is a block diagram of a heads-up display in accordance with the present invention connected to a surgical microscope;

FIG. 2 is a simulated view through a microscope in accordance with the present invention;

FIG. 3 is a system diagram showing an ophthalmic surgical system in accordance with the present invention;

FIG. 4 is a system diagram showing an alternate embodiment of an ophthalmic surgical system in accordance with the present invention; and

FIG. 5 is a system diagram of yet another alternate embodiment of an ophthalmic surgical system in accordance with the present invention.

### DETAILED DESCRIPTION OF THE PREFERRED EMBODIMENT

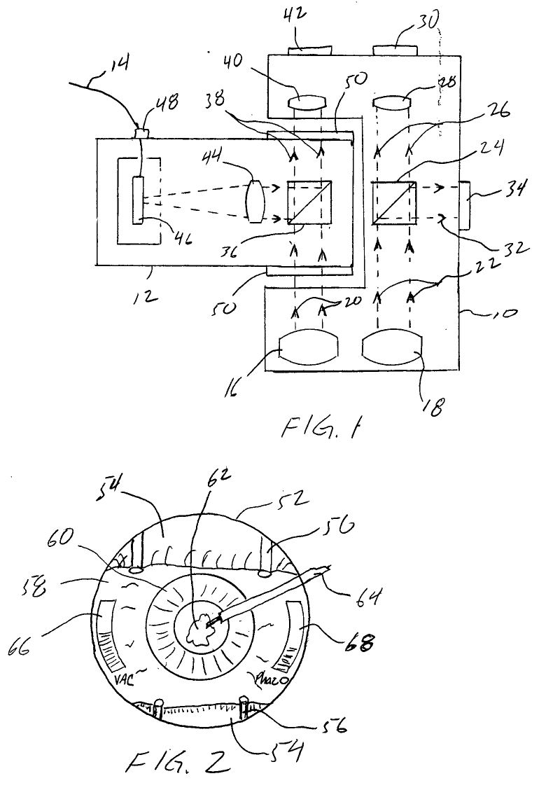

FIG. 1 shows a surgery-viewing device 10 for observing a surgical site (not shown). A heads-up display 12 is connected to the surgery-viewing device 10 and to a surgical console (not shown) via line 14 for displaying at least one surgical parameter to a user through the surgical viewing device 10.

Surgery viewing device 10 is preferably a surgical microscope as shown in block diagram form in FIG. 1, but may be other embodiments, such as those described below. Surgery viewing 10 is preferably a typical surgical microscope except that provision is made in the microscope for the attachment of a heads-up display 12 in accordance with the present invention. Microscope 10 observes a surgical site (not shown) through lenses 16 and 18. Culminated light passes through the lenses 16 and 18 in the direction of arrows 20 and 22. The light through lens 18 then passes through beam splitter 24, which splits the light, such that a portion of the light passes through as indicated by arrows 26 onto lens 28 and into eyepiece 30 for observation by a user. Beam splitter 24 also diverts a portion of the light as indicated at 32 to a separate port 34 for a second attachment. Port 34 may be used for attachment of a second observation viewer or may be connected to other equipment as is known in the art. The light passing through lens 16 along path 20 is split by beam splitter 36 such that a portion of the light passes along path 38 to lens 40 and on to eyepiece 42 for observance by a user. Beam splitter 36 also directs heads-up information onto path 38 through lens 44 through which an image is projected from display 46 into light path 38. Display 46 is connected through a standard connector 48 and line 14 to a surgical console (not shown) which supplies display 46 with surgical parameter information during surgery.

Display 46 may be any of a number of commercially available displays. Such factors in choosing display 46 include the size and resolution of display 46, as well as cost. Display 46 is preferably a small sized display so that heads-up display 12 may be sufficiently small not to encumber the surgery. Heads-up display 12 is connected to microscope 10 via connectors 50 in any known manner, such that the optical path 20 and 38 are not hindered.

The surgical parameters that may be displayed through heads-up display 12 include vacuum level, a phaco power low, surgery time, estimated intraocular pressure, and temperature. Other surgical parameters may also be displayed, depending on the type of surgery being performed and the surgical instruments being used in the surgery.

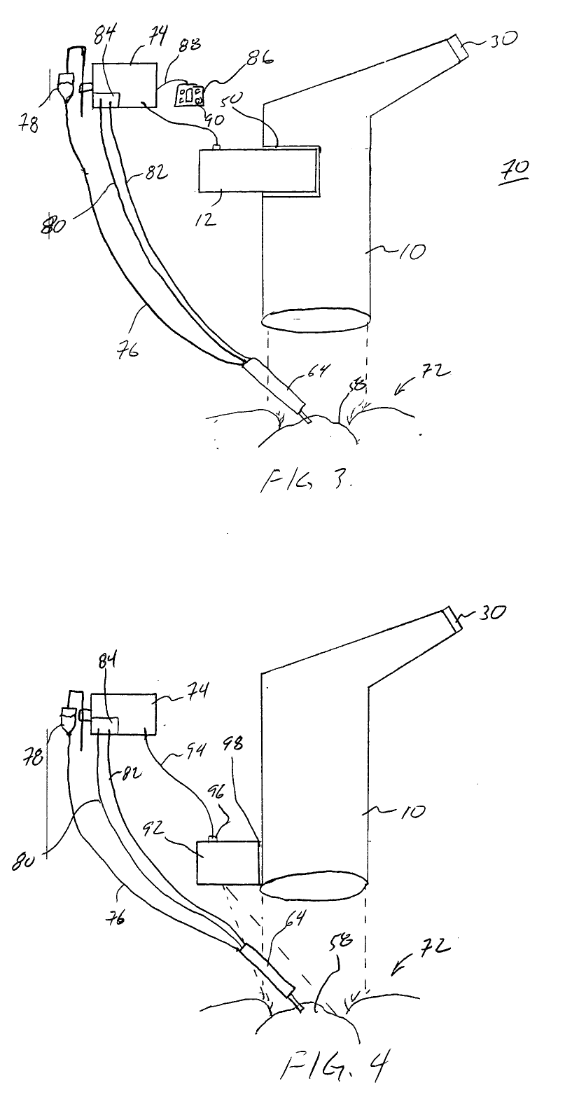

FIG. 2 shows a view of a surgical field 52 such as what might be seen through the system of FIG. 1. FIG. 2 shows an eyelid 54 being held open by a retractor 56 to reveal an eye 58 upon which cataract surgery is being performed. Eye 58 includes an iris 60 and a cataract 62, which is being removed by a phacoemulsification handpiece 64. Also being shown within view 52 is a bar graph of a vacuum level shown at 66 and a bar graph of a phaco power level shown at 68, which are projected through heads-up display 12 into microscope 10 as described above. In this way, the surgeon has critical information being displayed in the same view as the surgical site. In this way, his attention is not diverted to other displays and allows the surgeon to be aware of critical surgical parameters while not diverting his gaze or attention away from the surgery site.

FIG. 3 shows an ophthalmic surgical system 70 in accordance with the present invention. System 70 includes surgical viewing device 10 for observing a surgical site shown generally at 72 and which includes eye 58 and phacoemulsification handpiece 64 as described above with respect to FIG. 2. A surgical console 74 controls at least one surgical instrument, such as the phacoemulsification handpiece 64. Surgical console 74 also detects certain surgical parameters during surgery, such as described above. Heads-up display 12 is connected to each of the surgery-viewing device 10 and the surgical console 74 for displaying at least one of the surgical parameters to a user through the surgical viewing device 10. Phacoemulsification handpiece 64 is connected via irrigation line 76 to an irrigation bottle 78 as shown. Handpiece 64 is also connected to surgical console 74 through aspiration line 80 and power cable 82 to an aspiration device 84 which may be part of surgical console 74. A foot controller 86 is also preferably connected to surgical console 74 through line 88 and may include a button 90 for activating and deactivating the heads-up display 12. In addition, heads-up display 12 may be programmed to only be activated for a pre-determined period of time or to fade in and fade out over a pre-determined period of time. Such features may be desirable to a surgeon, depending on the type of surgery to be used in the information to be displayed to ensure that displayed surgical parameter information does not interfere with the surgeon's view of the surgical site. It is also possible for the heads-up display 12 to be activated via voice command as is known in the art.

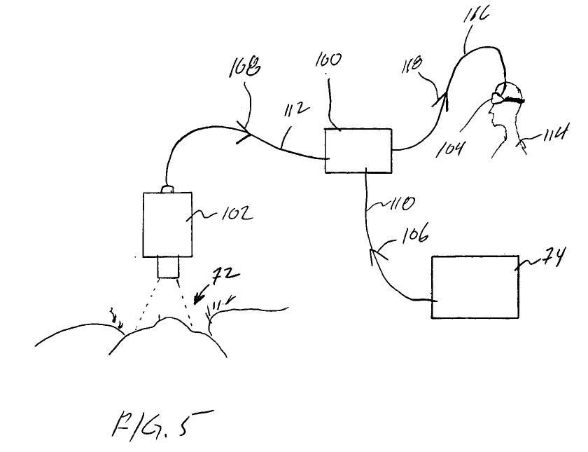

As described, surgery-viewing device 10 may be a surgical microscope as described above or may be other viewing means, such as described below with respect to FIGS. 4 and 5 are yet other viewing schemes known to those skilled in the art, but described.

Referring back to FIG. 1, heads-up display 12 includes an input cable 14 for attachment to a surgical console 74. Display 46 is connected to the input cable 14 via connector 48 for displaying images into the surgical viewing device 10. A lens 44 and beam splitter 36 combine to focus the display 46 and direct the display 46 images into the surgical viewing device 10 as described above.

Alternatively, a transparent display could be located in the image plane of eyepiece 42, in the surgical field of view, or a light emitting display could be located just outside the field stop of eyepiece 42, with the same effect.

Referring to FIG. 4, a heads-up display may alternatively include a laser writer 92 for connection to the surgical console 74 via line 94 through connector 96. Laser writer 92 is preferably connected via connector 98 to surgical viewing device 10 as shown. Connector 98 is simply attached to an outer housing of surgical viewing device 10 to allow laser writer 92 to be mounted onto device 10 as shown. Laser writer 92 then projects surgical parameter information from surgical console 74 onto the surgical site 72, such that a reflection from the projected surgical parameter information may be seen through the surgery-viewing device 10.

The view through surgery viewing device 10 of FIG. 4 is similar to that shown in FIG. 2, except that the graphical information 66 and 68 is transmitted to the user via reflection from the laser images reflected off of eye 58. Laser writer 92 is preferably similar to types of laser projectors known in the art that write information onto various surfaces including the eye.

The heads-up display may also include a mixer 100 as shown in the system as shown in FIG. 5. The mixer 100 is connected to each of a surgical console 74, which transmits information in the direction of arrow 106 to mixer 100 and camera 102, which transmits images to mixer 100 in the direction of arrow 108. Mixer 100 then overlays surgical parameters received via line 110 onto an image of the surgical site obtained by the camera 102 via line 112 such that a user 114 may view the surgical site 72 and the surgical parameters simultaneously on the display 104. The view through 104 is similar to that shown in FIG. 2 with respect to the other embodiments shown above. However, the view through display 104 is a virtual image, rather than an optically magnified image, as described above with respect to other embodiments. Those skilled in the art will appreciate that display 104 while shown connected to mixer 100 via line 116 and receiving information from mixer 100 in the direction of arrow 118 is shown as eye goggles. Other display devices may also be used, such as a viewing helmet of even a separate viewing display screen.

Thus has been shown three embodiments in accordance with the present invention wherein the heads-up display may be formed of a wide-variety of devices in order to transmit images of surgical parameters onto a view of a surgical site simultaneously for providing a surgeon with a convenient view of the surgical site and readily available surgical parameter information.

Another way to describe the heads-up display in accordance with the present invention is that a first connector is provided for connection to a surgery-viewing device and a second connector is provided for connection to a surgical console. The heads-up display then contains viewing means for transferring surgical parameters from the surgical console to the surgery viewing device, thereby enabling a surgical site and at least one of the surgical parameters to be viewed simultaneously.

1. An ophthalmic surgical system comprising:
a surgery-viewing device for observing a surgical site;
a surgical console for controlling at least one surgical instrument;
wherein the surgical console detects certain surgical parameters during surgery; and
a heads-up display connected to each of the surgery viewing device and the surgical console for displaying at least one of the surgical parameters to a user through the surgery-viewing device.

2. The system of claim 1 further including a foot controller for activating and deactivating the heads-up display.

3. The system of claim 1, wherein the heads-up display is activated for a predetermined time period.

4. The system of claim 1, wherein the surgical parameters include vacuum level, phaco power level, surgery time, estimated intraocular pressure, and temperature.

5. The system of claim 1, wherein the surgical viewing device is a surgical microscope.

6. The system of claim 1, wherein the surgery-viewing device is a camera and associated display device.

7. The system of claim 1, wherein the heads-up display includes
an input cable for attachment to the surgical console;
a display connected to the input cable for displaying images onto the surgical viewing device; and
a lens and beam splitter combination for focusing the display and directing the display images onto the surgery-viewing device.

8. The system of claim 1, wherein the heads-up display includes:
a laser writer for connection to the surgical console; and
wherein the laser writer projects surgical parameter information onto the surgical site such that a reflection of the projected surgical parameter information may be seen through the surgery-viewing device.

9. The system of claim 6, wherein the heads-up display includes:
a mixer connected to each of the surgical console and the camera for overlaying the surgical parameters onto an image of the surgical site obtained by the camera such that a user may view the surgical site and the surgical parameters simultaneously on the display.

10. A heads-up display comprising:
a first connector for connection to a surgery-viewing device;
a second connector for connection to a surgical console; and
viewing means for transferring surgical parameters from the surgical console to the surgery-viewing device, thereby enabling a surgical site and at least one of the surgical parameters to be viewed simultaneously.

11. The invention of claim 10 further including a connector for connection to a foot controller for activating and deactivating the heads-up display.

12. The invention of claim 10, wherein the surgical parameters include vacuum level, phaco power level, surgery time, estimated intraocular pressure, and temperature.

13. The invention of claim 10, wherein the surgical viewing device is a surgical microscope.

14. The system of claim 10, wherein the surgery-viewing device is a camera and associated display device.

15. The system of claim 10, wherein the heads-up display includes
an input cable for attachment to the surgical console;
a display connected to the input cable for displaying images onto the surgical viewing device; and
a lens and beam splitter combination for focusing the display and directing the display images onto the surgery-viewing device.

16. The system of claim 10, wherein the heads-up display includes:
a laser writer for connection to the surgical console; and
wherein the laser writer projects surgical parameter information onto the surgical site such that a reflection of the projected surgical parameter information may be seen through the surgery-viewing device.

17. The system of claim 14, wherein the heads-up display includes:
a mixer connected to each of the surgical console and the camera for overlaying the surgical parameters onto an image of the surgical site obtained by the camera such that a user may view the surgical site and the surgical parameters simultaneously on the display.
